(19) United States
(12) Patent Application Publication
(10) Pub. No.: US 2026/0207915 A1
(43) Pub. Date: Jul. 23, 2026

(54) Devices for shunting blood

(71) Applicant: Inspire M.D Ltd., Tel Aviv (IL)

(72) Inventor: Stefan Schreck, Duvall, WA (US)

(21) Appl. No.: 19/567,449

(22) Filed: Mar. 16, 2026

Related U.S. Application Data
(62) Division of application No. 17/889,423, filed on Aug. 17, 2022.

(51) Int. Cl. A61M 39/06; A61M 29/02; A61M 39/02; A61M 39/10

(52) U.S. Cl. CPC A61M 39/0613; A61M 29/02; A61M 39/0208; A61M 39/1011; A61M 2029/025; A61M 2039/0241; A61M 2039/0258; A61M 2039/062

(57) ABSTRACT

A kit for use with a hub including a front port, which is connected to a sheath, and a back port, which includes a hemostasis valve, includes a tube and an adapter configured to connect to the tube and configured for insertion through the hemostasis valve such that, subsequently to an insertion of the sheath into a blood vessel, blood flows between the blood vessel and the tube via the adapter. Other embodiments are also described.

### CROSS-REFERENCE TO RELATED APPLICATIONS

The present application is a divisional of U.S. application Ser. No. 17/889,423, filed Aug. 17, 2022.

### FIELD OF THE INVENTION

The present invention is related to the field of medical devices, especially devices for shunting blood from an artery to a vein.

### BACKGROUND

U.S. Pat. No. 8,545,432 describes a retrograde flow system for treating an artery. The system includes an arterial access device adapted to be introduced into an artery and receive blood flow from the artery. A shunt is fluidly connected to the arterial access device, wherein the shunt provides a pathway for blood to flow from the arterial access device to a return site. A flow control assembly is coupled to the shunt and is adapted to regulate blood flow through the shunt between at least a first blood flow state and at least a second blood flow state. A shut-off valve assembly automatically blocks fluid flow through the shunt in response to injection of the fluid into the arterial access device.
U.S. Pat. No. 7,063,685 describes a hemostasis valve that is closed when not accessed, but which provides an unobstructed fluid pathway when accessed by a luer fitting/connector. An auto-closure valve within the hemostasis valve housing is flush with a top surface thereof. When accessed with a connector or luer fitting, the valve opens completely, allowing an unobstructed high flow fluid path.

### SUMMARY OF THE INVENTION

There is provided, in accordance with some embodiments of the present invention, an apparatus including a hub including a front port, a side port, and a back port including a hemostasis valve. The apparatus further includes a sheath extending from the hemostasis valve through the front port and shaped to define one or more lateral openings within the hub. The sheath is configured for insertion into a blood vessel such that, subsequently to the insertion, a rate of flow of blood between the side port and the blood vessel via the lateral openings is controllable using an instrument passing through the hemostasis valve and into the sheath such that the instrument closes at least a portion of the lateral openings.

In some embodiments, a cross-sectional area of a lumen of the side port is at least 3.8 mm².

In some embodiments, a total area of the lateral openings is at least as large as an area of the side port.

In some embodiments, the sheath includes:
a back sheath, which is disposed within the hub and is shaped to define the lateral openings; and
a front sheath, which is connected to the back sheath and is configured for insertion into the blood vessel.

In some embodiments, the sheath is connected to the back port.

In some embodiments, the apparatus further includes a tube configured to connect to the side port.

In some embodiments, the apparatus further includes the instrument.

In some embodiments, the instrument includes a dilator configured to facilitate the insertion by dilating the blood vessel.

In some embodiments, the instrument includes one or more markings configured to indicate a size of the portion of the lateral openings that is closed by the instrument, by virtue of the markings progressively entering the hub as the instrument is pushed through the hemostasis valve.

In some embodiments, the openings are arranged in one or more rows.

In some embodiments, the openings include one or more slots.

In some embodiments, the apparatus further includes a blood filter disposed between the lateral openings and the side port such that the blood flows through the blood filter.

There is further provided, in accordance with some embodiments of the present invention, a method including inserting a sheath, which extends from a hemostasis valve at a back port of a hub through a front port of the hub, into a blood vessel, the sheath being shaped to define one or more lateral openings within the hub. The method further includes, subsequently to inserting the sheath, controlling a rate of flow of blood between a side port of the hub and the blood vessel via the lateral openings, by passing an instrument through the hemostasis valve and into the sheath such that the instrument closes at least a portion of the lateral openings.

There is further provided, in accordance with some embodiments of the present invention, a kit for use with a hub including a front port, which is connected to a sheath, and a back port, which includes a hemostasis valve. The kit includes a tube, and an adapter configured to connect to the tube and configured for insertion through the hemostasis valve such that, subsequently to an insertion of the sheath into a blood vessel, blood flows between the blood vessel and the tube via the adapter.

In some embodiments, the adapter includes one or more barbs configured to inhibit the adapter from exiting the hub subsequently to the insertion of the adapter.

In some embodiments, the adapter includes:
a straight connector, including:
   a front end, configured for insertion through the hemostasis valve; and
   a back end; and
a tube connector, which includes a front port connected to the back end of the straight connector and is configured to connect to the tube.

In some embodiments, the tube connector further includes a back port configured to connect to the tube.

In some embodiments, the tube connector further includes:
a back port, including another hemostasis valve; and
one or more side ports, at least one of which is configured to connect to the tube.

In some embodiments, the tube connector includes a T-connector.

In some embodiments, the tube connector includes a Y-connector.

In some embodiments, the kit further includes another adapter configured for insertion into another hub connected to another sheath configured for insertion into another blood vessel.

In some embodiments, the kit further includes:
another hub including another front port, a side port, and another back port including another hemostasis valve; and
another sheath extending from the other hemostasis valve through the other front port and shaped to define one or more lateral openings within the other hub,
   the other sheath being configured for insertion into another blood vessel such that, subsequently to the insertion, a rate of flow of the blood between the side port and the other blood vessel via the lateral openings is controllable using an instrument passing through the other hemostasis valve and into the other sheath such that the instrument closes at least a portion of the lateral openings.

There is further provided, in accordance with some embodiments of the present invention, an apparatus including a sheath. The apparatus further includes a hub, including a front port, which is connected to the sheath, and a back port including a hemostasis valve. The apparatus further includes an adapter passing through the hemostasis valve and a tube connected to the adapter such that, subsequently to an insertion of the sheath into a blood vessel, blood flows between the blood vessel and the tube via the adapter.

There is further provided, in accordance with some embodiments of the present invention, a method including inserting an adapter, which is connected to a tube, through a hemostasis valve at a back port of a hub. The method further includes inserting a sheath, which is connected to a front port of the hub, into a blood vessel such that blood flows between the blood vessel and the tube via the adapter.

In some embodiments,
the adapter is a first adapter, the hub is a first hub, the hemostasis valve is a first hemostasis valve, the sheath is a first sheath, and the blood vessel is a first blood vessel, and
the method further includes:
   inserting a second adapter through a second hemostasis valve at a back port of a second hub;
   inserting a second sheath, which is connected to a front port of the second hub, into a second blood vessel; and
   connecting the tube to the second hub such that the blood flows between the first blood vessel and the second blood vessel via the tube.

In some embodiments, connecting the tube to the second hub includes connecting the tube to the second hub via a device configured to perform a function selected from the group of functions consisting of: filtering the blood, indicating a flow rate of the blood, and facilitating control of the flow rate.

In some embodiments,
the hub is a first hub, the hemostasis valve is a first hemostasis valve, the sheath is a first sheath, and the blood vessel is a first blood vessel, and
the method further includes:
   inserting a second sheath, which extends from a second hemostasis valve at a back port of a second hub through a front port of the second hub, into a second blood vessel, the sheath being shaped to define one or more lateral openings within the second hub; and
connecting the tube to the second hub such that the blood flows between the first blood vessel and the second blood vessel via the tube and the lateral openings.

The present invention will be more fully understood from the following detailed description of embodiments thereof, taken together with the drawings, in which:

### BRIEF DESCRIPTION OF THE DRAWINGS

FIG. 1A is a schematic illustration of an introducer apparatus, in accordance with some embodiments of the present invention;

FIG. 1B is a schematic illustration of a cross-section through the introducer apparatus of FIG. 1A, in accordance with some embodiments of the present invention;

FIGS. 2A-B are schematic illustrations of a sheath, in accordance with some embodiments of the present invention;

FIG. 3 is a schematic illustration of a technique for controlling a rate of blood flow through a hub, in accordance with some embodiments of the present invention;

FIG. 4 is a schematic illustration of an introducer apparatus, in accordance with some embodiments of the present invention;

FIGS. 5A-B are schematic illustrations of an adapter connected to a tube, in accordance with some embodiments of the present invention;

FIGS. 6A-C are schematic illustrations of techniques for shunting blood, in accordance with some embodiments of the present invention; and

FIG. 7 is a schematic illustration of a kit for use with a conventional introducer hub, in accordance with some embodiments of the present invention.

### DETAILED DESCRIPTION OF EMBODIMENTS

### Overview

In some procedures, an introducer apparatus, also referred to herein simply as an “introducer,” provides access to a blood vessel of a subject. An introducer comprises a sheath connected to an introducer hub. Optionally, following the percutaneous insertion of the sheath into the blood vessel, other tools and devices may be passed through the sheath and into the blood vessel via the hub.

During a procedure to clear an occlusive lesion from an artery, such as a carotid artery, the flow of blood toward the lesion may be stopped so as to prevent any emboli from being carried downstream. While the downstream flow through the artery is stopped, blood reaching the area of the lesion via other arteries may be diverted through the sheath and introducer hub, such that any debris generated by the procedure may be safely removed from the subject's body. To prevent an excessive loss of blood, the blood may be shunted, through a filter, to a vein of the subject. For example, the blood may be shunted to a femoral, radial, or jugular vein.

In a conventional introducer, a front port of the hub is connected to the back end of the sheath, and a side port of the hub is connected to a tube via which the blood may be shunted. While the blood flows through the side port, a guidewire, a stent, and/or any other tool or device may be passed through a back port of the hub, which typically comprises a hemostasis valve.

One challenge with conventional introducers is that the rate of blood flow through the hub cannot be controlled.

To address this challenge, embodiments of the present invention provide an improved introducer, which may be used at either the arterial side or the venous side of a shunt. In this introducer, the sheath does not terminate at the front port of the hub, but rather, extends through the hub to the back port of the hub. The portion of the sheath inside the hub is shaped to define one or more lateral openings through which the blood may flow. By inserting an instrument, such as a dilator or an obturator, through the back port of the hub and into the sheath, the openings may be selectively occluded, thereby reducing the flow rate of the blood. Advantageously, the instrument may comprise markings that indicate, to the physician, the extent to which the openings are occluded.

Another challenge with conventional introducers is that the rate of blood flow may be restricted by the relatively small lumen of the side port.

The improved introducer described above may address this challenge by providing a side port having a widened lumen.

In addition, embodiments of the present invention provide an adapter configured to increase the flow rate through a conventional hub. The adapter is inserted through the hemostasis valve of the back port of the hub, and a tube for blood flow is connected to the adapter. The adapter thus facilitates using the wider back port of the hub, rather than the side port, for blood flow.

The adapter may be used at either the arterial side or the venous side of a shunt. At the arterial side, the adapter typically comprises one or more side ports, one of which is connected to the tube, and the back port of the adapter is used for the passage of tools and devices. At the venous side, the tube may be connected to the back port of the adapter, and the adapter need not necessarily comprise any side ports.

### Introducer With Flow-Rate Control

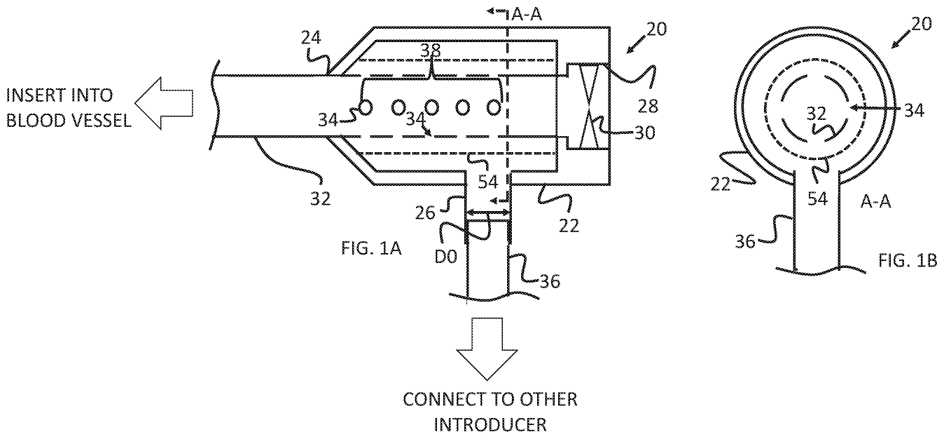

Reference is initially made to FIG. 1A, which is a schematic illustration of an introducer apparatus 20, in accordance with some embodiments of the present invention. Reference is further made to FIG. 1B, which is a schematic illustration of a cross-section A-A through apparatus 20, in accordance with some embodiments of the present invention.

Apparatus 20 comprises a hub 22 comprising a front port 24, a side port 26, and a back port 28 comprising a hemostasis valve 30. Each of the ports may comprise a protrusion from the body of the hub as in the case of side port 26 in FIG. 1A, a chamber recessed within the body of the hub as in the case of back port 28 in FIG. 1A, or a simple opening as in the case of front port 24 in FIG. 1A.

Apparatus 20 further comprises a sheath 32 extending from the hemostasis valve through the front port and shaped to define one or more lateral openings 34 within hub 22. For example, for each opening 34, a vector normal to the opening may be perpendicular to the longitudinal axis of sheath 32 at the opening. Sheath 32 may have any suitable diameter, such as 6-18 Fr. Typically, the back end of sheath 32 is bonded to back port 28.

Sheath 32 is configured for insertion into a blood vessel. As further described below with reference to FIG. 3, subsequently to the insertion of the sheath into the blood vessel, the rate of flow of blood between side port 26 and the blood vessel via openings 34 may be controlled using an instrument passing through hemostasis valve 30 and into the sheath such that the instrument closes at least a portion of the openings.

Typically, the cross-sectional area of the lumen of side port 26 is larger than in a conventional introducer. For example, the inner diameter D0 of the side port may be at least 2 mm and/or the cross-sectional area of the lumen may be at least 3.1 mm².

Typically, the total area of openings 34 is at least as large as the cross-sectional area of the side-port lumen, such that the rate of blood flow is not limited by the openings. For example, the total area of the openings may be 20-100% greater than the area of the side-port lumen. As a specific example, the area of the side-port lumen may be 4 mm², and the total area of the openings may be 5-8 mm².

Typically, during a procedure on an artery, the sheath is inserted into a vein so as to facilitate the shunting of blood from the artery to the vein. As a specific example, during a procedure on a carotid artery, the sheath may be inserted into a femoral, radial, or jugular vein so as to facilitate the shunting of blood from the carotid artery to the vein.

In other embodiments, the sheath is inserted into the artery. In such embodiments, a separate, conventional introducer may be used to introduce tools into the artery, such that back port 28 of apparatus 20 may be reserved for controlling the rate of blood flow.

Typically, apparatus 20 further comprises a tube 36 configured to connect to side port 26. For example, tube 36 may be permanently connected to the side port, e.g., by virtue of being bonded to the inner or outer wall of the side port. Alternatively, the tube may be configured to reversibly connect to the side port, e.g., via a Luer-lock connection.

Tube 36 is configured to carry blood to or from hub 22.

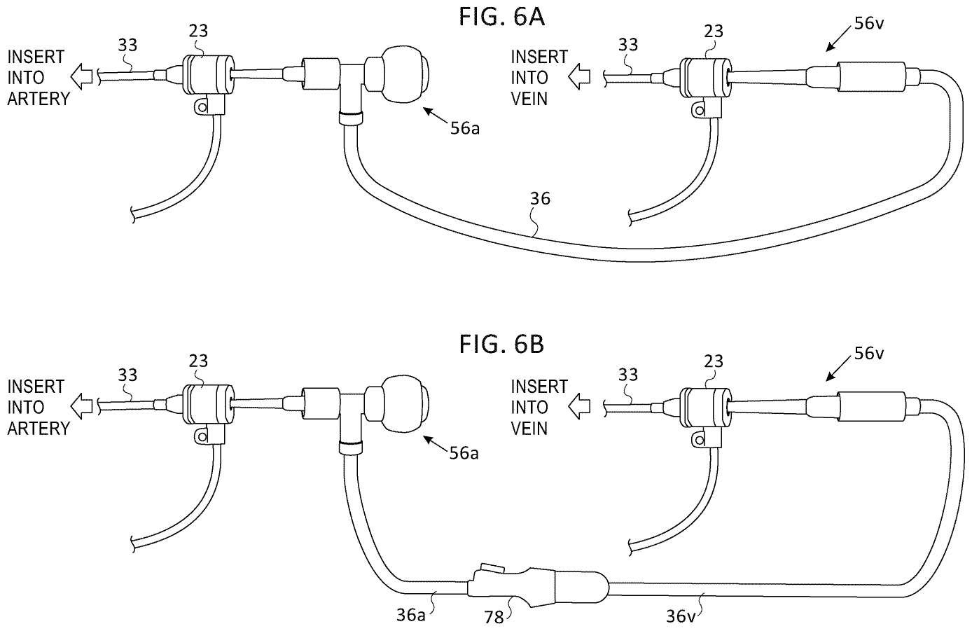

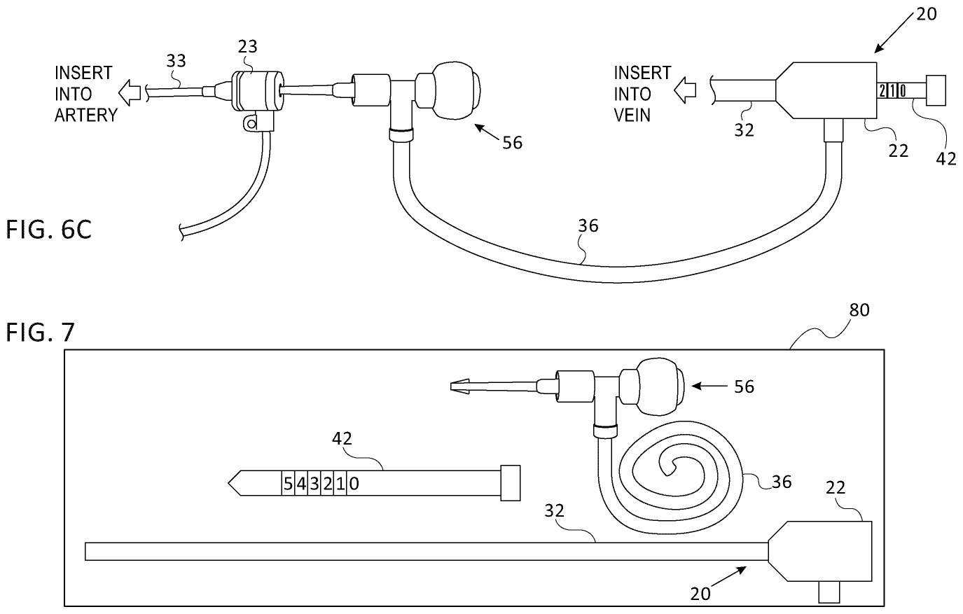

For example, for embodiments in which sheath 32 is inserted into a vein, tube 36 may be connected to another introducer apparatus comprising another sheath inserted into an artery, such that the tube carries blood from the artery to hub 22. (An example of such embodiments is described below with reference to FIG. 6C.) Alternatively, for embodiments in which sheath 32 is inserted into an artery, tube 36 may be connected to another introducer apparatus comprising another sheath inserted into a vein, such that the tube carries blood from the hub to the vein. In each of the examples above, the tube may be connected to the other introducer directly or via another tube, and the connection may be permanent or reversible.

Alternatively, tube 36 may carry blood from the hub to a collection bag.

In some embodiments, apparatus 20 further comprises a blood filter 54 disposed between the lateral openings and the side port such that the blood flows through blood filter 54. For example, hub 22 may contain a cylindrical blood filter 54 that surrounds sheath 32.

Alternatively or additionally, side port 26 may contain a blood filter.

In some embodiments, at least a portion of hub 22 is transparent. One or more moveable objects, such as beads, are disposed within the hub such that the moveable objects may be viewed through the transparent portion of the hub. The moveable objects are configured to move in response to the flow of blood through the hub, thereby indicating the flow to the physician. The moveable objects may have any of the features described in U.S. Patent application Ser. No. 17/576,953, whose disclosure is incorporated herein by reference.

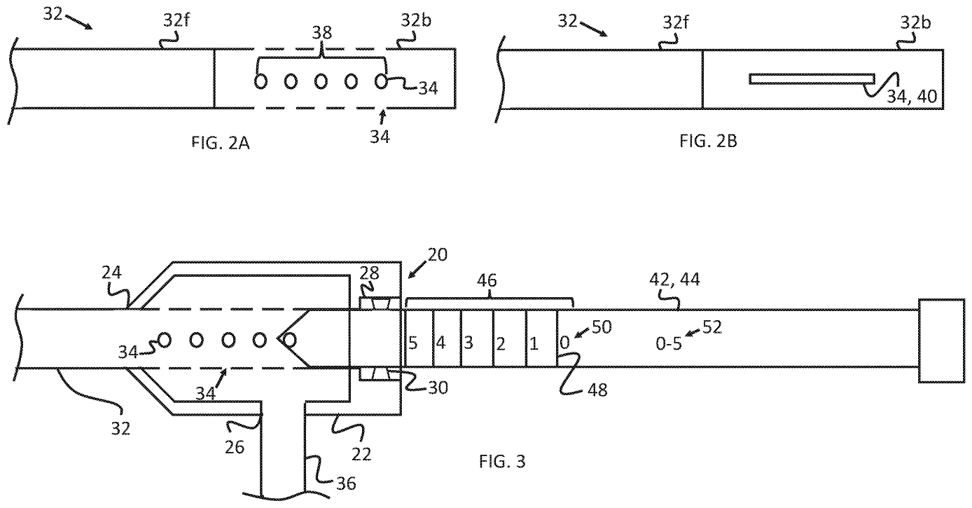

Reference is now additionally made to FIGS. 2A-B, which are schematic illustrations of sheath 32, in accordance with some embodiments of the present invention.

In some embodiments, openings 34 are arranged in one or more rows 38, such as multiple rows 38 distributed around the circumference of sheath 32. For example, as shown in FIGS. 1A and 2A, openings 34 may be arranged in four rows distributed around the circumference of the sheath. Each row may include any number of openings, such as between two and ten openings.

In such embodiments, openings 34 may be circular or may have any other suitable shape.

Alternatively or additionally, as shown in FIG. 2B, the openings may include one or more slots 40, such as multiple (e.g., four) slots 40 distributed around the circumference of sheath 32. Typically, in such embodiments, the longitudinal axis of each slot is parallel to the longitudinal axis of the sheath at the slot.

In some embodiments, as shown in FIGS. 2A-B, sheath 32 comprises a back sheath 32b, which is disposed within hub 22 and is shaped to define openings 34, and a front sheath 32f. Front sheath 32f is connected (e.g., bonded) to back sheath 32b; in particular, the back end of the front sheath is connected to the front end of the back sheath, such that blood may flow between the front sheath and the back sheath. The front sheath, which is typically more flexible than the back sheath, is configured for insertion into the blood vessel.

In some embodiments, hub 22 is manufactured by molding a polymer such as acrylonitrile butadiene styrene (ABS) or a polycarbonate. In such embodiments, back sheath 32b may be molded together with the hub, such that the back sheath is integral with the hub. Alternatively, regardless of the material from which the hub is manufactured and regardless of the technique used to manufacture the hub, back sheath 32b—or the entire sheath, for embodiments in which the sheath does not comprise connected front and back sections-may be manufactured separately from the hub and then connected (e.g., bonded) to back port 28. For example, back sheath 32b may be manufactured by extruding a polymer such as polyethylene or polyether block amide (PEBA) and then forming openings 34 in the extrusion.

Reference is now made to FIG. 3, which is a schematic illustration of a technique for controlling a rate of blood flow through hub 22, in accordance with some embodiments of the present invention.

To control the rate of flow between side port 26 and the blood vessel into which sheath 32 is inserted, an instrument 42 is passed through hemostasis valve 30. As instrument 42 is passed further into the sheath, the instrument occludes more of openings 34, thereby restricting blow flow between the side port and the sheath. Conversely, as the instrument is withdrawn, more of the openings are opened, such that the flow rate through the openings is increased. In some embodiments, instrument 42 is provided, together with the other components of apparatus 20, in a kit.

Typically, to facilitate controlling the rate of blood flow between the side port and the sheath as described above, the outer diameter of the instrument is approximately the same as the inner diameter of sheath 32.

In some embodiments, instrument 42 comprises a dilator 44 configured to facilitate the insertion of the sheath into the blood vessel by dilating the blood vessel. In other words, subsequently to dilating the blood vessel, dilator 44 may be used to control the flow rate. For example, the dilator may be passed through the sheath, via back port 28 and hemostasis valve 30, until the dilator punctures the blood vessel. Subsequently, the dilator may be withdrawn until at least a portion of openings 34 is not occluded. Subsequently, the dilator may be further withdrawn or advanced so as to control the flow rate through openings 34.

In other embodiments, a separate instrument 42 is used. For example, after completely withdrawing the dilator from the hub, an obturator-which is shorter, and thus easier to use, than the dilator-may be inserted into the hub and used for flow control.

Typically, instrument 42 comprises one or more markings 46 configured to indicate the size of the portion of the lateral openings that is closed by the instrument, by virtue of markings 46 progressively entering the hub as the instrument is pushed through the hemostasis valve. In other words, the markings are placed such that when the openings are not occluded at all, all the markings are outside the hub, but as the occlusion is increased, more of the markings enter the hub. Thus, the physician may readily ascertain the extent of the occlusion based on the extent to which the markings are visible.

For example, markings 46 may comprise a series of arcs 48 placed at different respective positions along the longitudinal axis of the instrument, such that the number of visible arcs 48 indicates the extent to which the openings are occluded. (Each arc 48 may be closed, thus defining a circle.) For embodiments in which openings 34 are arranged in rows, the arcs may be placed such that the number of visible arcs equals the number of openings in each row that are not occluded by the instrument.

Alternatively or additionally to arcs 48, markings 46 may comprise alphanumeric markings, such as numbers 50, such that the frontmost visible marking indicates the amount of occlusion. For embodiments in which openings 34 are arranged in rows, the frontmost visible marking may indicate the number of openings in each row that are not occluded by the instrument.

In some embodiments, a scale 52, which facilitates interpreting the markings, is also marked on the instrument. For example, for embodiments in which the markings comprise numbers 50, scale 52 may include the minimum and maximum number separated by a hyphen from one another.

### Adapter for Increased Flow Rate

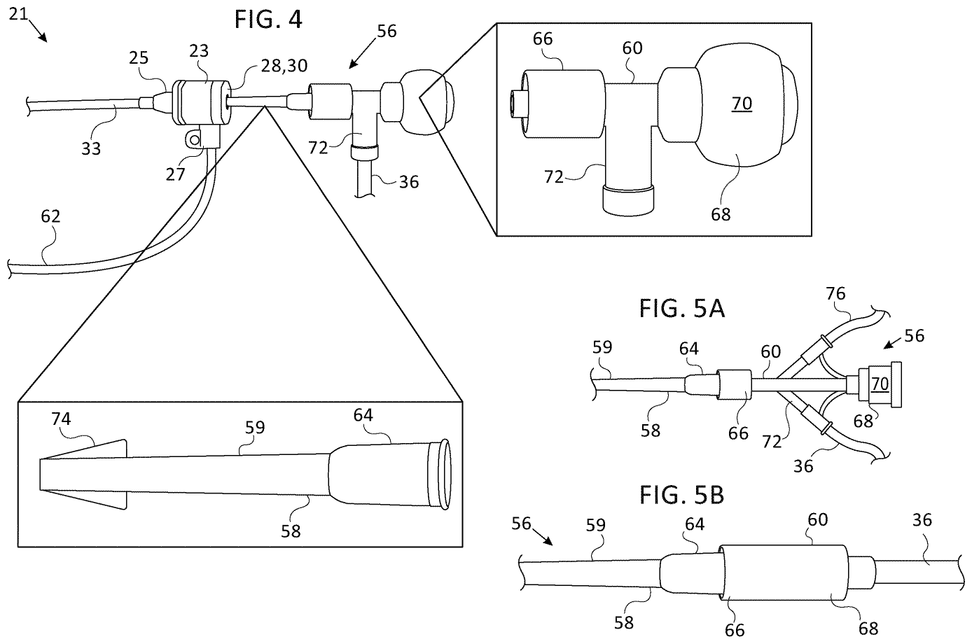

Reference is now made to FIG. 4, which is a schematic illustration of a conventional introducer apparatus 21 with an adapter 56, in accordance with some embodiments of the present invention. Reference is further made to FIGS. 5A-B, which are schematic illustrations of an adapter 56 connected to tube 36, in accordance with some embodiments of the present invention.

Apparatus 21 comprises a hub 23 and a sheath 33, which have conventional features and are connected to one another in a conventional way. In particular, sheath 33 is not shaped to define any lateral openings and does not pass through hub 23; rather, sheath 33 is connected to a front port 25 of the hub.

Adapter 56 is configured to facilitate the flow of blood between hub 23 and tube 36 via back port 28 of the hub. In particular, the adapter is configured to connect permanently or reversibly to the tube and is configured for insertion through hemostasis valve 30 such that, subsequently to the insertion of sheath 33 into a blood vessel, blood flows between the blood vessel and tube 36 via the adapter. Typically, to facilitate this flow, the diameter of the lumen of adapter 56 at the narrowest portion of the lumen is at least 2 mm.

Typically, adapter 56 comprises a straight connector 58 and a tube connector 60, which is configured to connect to tube 36. Straight connector 58 comprises a front end 59, configured for insertion through the hemostasis valve of hub 23, and a back end 64. Tube connector 60 comprises a front port 66 connected to back end 64. Typically, tube connector 60 further comprises a back port 68.

Typically, front port 66 is connected to back end 64 via a Luer-lock connection. For example, front port 66 may comprise a male Luer lock, and back end 64 may comprise a female Luer lock connected to the male Luer lock. Alternatively, front port 66 may be bonded to back end 64.

In other embodiments, adapter 56 is manufactured as a single integrated connector, which may comprise features of straight connector 58 and tube connector 60.

In some embodiments, as shown in FIG. 5B, back port 68 is configured to connect to tube 36, e.g., by virtue of being bonded or Luer-locked to the tube. Typically, in such embodiments, the tube connector does not comprise any side ports, and sheath 33 is inserted into a vein.

In other embodiments, as shown in FIGS. 4 and 5A, tube connector 60 comprises one or more side ports 72, at least one of which is configured to connect to tube 36, e.g., by virtue of being bonded or Luer-locked to the tube. (Optionally, the side port may connect to the tube via an aspiration port.) Typically, in such embodiments, back port 68 comprises another hemostasis valve 70, and sheath 33 is inserted into an artery. A guidewire, a catheter, a stent, and/or any other tool or device may be passed through back port 68.

For example, as shown in FIG. 4, the tube connector may comprise a T-connector, which comprises a single side port 72 perpendicular to the axis passing between front port 66 and back port 68.

Alternatively, as shown in FIG. 5A, the tube connector may comprise a Y-connector, which comprises one or more side ports 72 at an oblique angle with respect to the axis passing between front port 66 and back port 68. For example, the Y-connector may comprise two side ports. One of the side ports may be connected to tube 36, and the other side port may be connected to another tube 76. Saline, contrast material, and/or any other fluid may be inserted into the blood vessel via tube 76.

As yet another alternative, the tube connector may comprise a series of two T-connectors or two Y-connectors, or a Y-connector connected to a T-connector. Typically, in such embodiments, the back port of the front member of the series comprises a Luer lock for locking to the back member of the series, while the back port of the back member of the series comprises hemostasis valve 70.

In some embodiments, adapter 56 comprises one or more barbs 74 configured to inhibit the adapter from exiting hub 23 subsequently to the insertion of the adapter into the hub. For example, as shown in FIG. 4, for embodiments in which the adapter comprises straight connector 58, the straight connector may comprise barbs 74. Each barb 74 is shaped such that the barb generates relatively little resistance when passed forward through hemostasis valve 30 but significant resistance when pulled backward. For example, each barb may comprise a triangular protrusion that is wider at the back than at the front, such that the valve resists the backward sliding of the protrusion therethrough.

In some embodiments, another tube 62 is connected to a side port 27 of the hub. Saline, contrast material, and/or any other fluid may be inserted into the blood vessel via tube 62.

Reference is now made to FIGS. 6A-C, which are schematic illustrations of techniques for shunting blood, in accordance with some embodiments of the present invention.

As described above with reference to FIG. 1A, tube 36 may facilitate shunting blood between two blood vessels.

For example, one adapter 56 may be inserted into a first introducer inserted into a first blood vessel, and another adapter may be inserted into a second introducer inserted into a second blood vessel. Blood may then flow between the two blood vessels via tube 36.

As a specific example, as shown in FIG. 6A, tube 36 may connect at one end to an arterial-side adapter 56a, comprising the adapter of FIG. 4 or FIG. 5A for example, and at the other end to a venous-side adapter 56v, comprising the adapter of FIG. 5B for example. Alternatively, as shown in FIG. 6B, the two adapters may be connected to one another via a device 78 for filtering the blood, indicating the rate of blood flow, and/or controlling the rate of blood flow. In particular, arterial-side adapter 56a may be connected directly or via an arterial-side tube 36a to the inlet port of device 78, and venous-side adapter 56v may be connected directly or via a venous-side tube 36v to the outlet port of device 78. (Each connection may be permanent, e.g., via bonding, or reversible, e.g., via a Luer lock.) Example embodiments of device 78 are described, for example, in International Application PCT/IB2022/052688 and U.S. patent application Ser. No. 17/576,953, whose respective disclosures are incorporated herein by reference.

Alternatively, as shown in FIG. 6C, an adapter 56 and an introducer apparatus 20 may be connected to one another via tube 36. An advantage of such embodiments is that adapter 56 and the widened side port of apparatus 20 facilitate an increased flow rate, yet the flow rate can be reduced, if necessary, using instrument 42. Typically, in such embodiments, adapter 56 is inserted into an arterial-side introducer, and sheath 32 of apparatus 20 is inserted into a vein.

Alternatively to facilitating the shunting of blood, tube 36 may carry blood from an arterial-side adapter to a collection bag.

Reference is now made to FIG. 7, which is a schematic illustration of a kit for use with a conventional introducer hub, in accordance with some embodiments of the present invention.

Typically, an arterial-side adapter and/or a venous-side adapter is provided, together with tube 36, in a kit 80. (The tube may be preconnected to at least one of the adapters.) Kit 80 may further comprise additional components such as apparatus 20, instrument 42, device 78 (FIG. 6B), and/or a conventional introducer. Thus, advantageously, kit 80 may facilitate implementing any suitable shunting technique, such as any of the techniques illustrated in FIGS. 6A-C.

It will be appreciated by persons skilled in the art that the present invention is not limited to what has been particularly shown and described hereinabove. Rather, the scope of embodiments of the present invention includes both combinations and subcombinations of the various features described hereinabove, as well as variations and modifications thereof that are not in the prior art, which would occur to persons skilled in the art upon reading the foregoing description. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A kit for use with a hub including a front port, which is connected to a sheath, and a back port, which includes a hemostasis valve, the kit comprising:
a tube; and
an adapter configured to connect to the tube and configured for insertion through the hemostasis valve such that, subsequently to an insertion of the sheath into a blood vessel, blood flows between the blood vessel and the tube via the adapter.

2. The kit according to claim 1, wherein the adapter comprises one or more barbs configured to inhibit the adapter from exiting the hub subsequently to the insertion of the adapter.

3. The kit according to claim 1, wherein the adapter comprises:
a straight connector, comprising:
a front end, configured for insertion through the hemostasis valve; and
a back end; and
a tube connector, which comprises a front port connected to the back end of the straight connector and is configured to connect to the tube.

4. The kit according to claim 3, wherein the tube connector further comprises a back port configured to connect to the tube.

5. The kit according to claim 3, wherein the tube connector further comprises:
a back port, comprising another hemostasis valve; and
one or more side ports, at least one of which is configured to connect to the tube.

6. The kit according to claim 3, wherein the tube connector comprises a T-connector.

7. The kit according to claim 3, wherein the tube connector comprises a Y-connector.

8. The kit according to claim 1, further comprising another adapter configured for insertion into another hub connected to another sheath configured for insertion into another blood vessel.

9. The kit according to claim 1, further comprising:
another hub comprising another front port, a side port, and another back port comprising another hemostasis valve; and
another sheath extending from the other hemostasis valve through the other front port and shaped to define one or more lateral openings within the other hub,
the other sheath being configured for insertion into another blood vessel such that, subsequently to the insertion, a rate of flow of the blood between the side port and the other blood vessel via the lateral openings is controllable using an instrument passing through the other hemostasis valve and into the other sheath such that the instrument closes at least a portion of the lateral openings.

10. Apparatus, comprising:
a sheath;
a hub, comprising a front port, which is connected to the sheath, and a back port comprising a hemostasis valve;
an adapter passing through the hemostasis valve; and
a tube connected to the adapter such that, subsequently to an insertion of the sheath into a blood vessel, blood flows between the blood vessel and the tube via the adapter.

11. A method, comprising:
inserting an adapter, which is connected to a tube, through a hemostasis valve at a back port of a hub; and
inserting a sheath, which is connected to a front port of the hub, into a blood vessel such that blood flows between the blood vessel and the tube via the adapter.

12. The method according to claim 11,
wherein the adapter is a first adapter, the hub is a first hub, the hemostasis valve is a first hemostasis valve, the sheath is a first sheath, and the blood vessel is a first blood vessel, and
wherein the method further comprises:
inserting a second adapter through a second hemostasis valve at a back port of a second hub;
inserting a second sheath, which is connected to a front port of the second hub, into a second blood vessel; and
connecting the tube to the second hub such that the blood flows between the first blood vessel and the second blood vessel via the tube.

13. The method according to claim 12, wherein connecting the tube to the second hub comprises connecting the tube to the second hub via a device configured to perform a function selected from the group of functions consisting of: filtering the blood, indicating a flow rate of the blood, and facilitating control of the flow rate.

14. The method according to claim 11,
wherein the hub is a first hub, the hemostasis valve is a first hemostasis valve, the sheath is a first sheath, and the blood vessel is a first blood vessel, and
wherein the method further comprises:
inserting a second sheath, which extends from a second hemostasis valve at a back port of a second hub through a front port of the second hub, into a second blood vessel, the sheath being shaped to define one or more lateral openings within the second hub; and
connecting the tube to the second hub such that the blood flows between the first blood vessel and the second blood vessel via the tube and the lateral openings.
